(19) United States
(12) Patent Application Publication
(10) Pub. No.: US 2020/0268967 A1
(43) Pub. Date: Aug. 27, 2020

(54) Infuser

(71) Applicant: Steam Tech, LLC, Denver, CO (US)

(72) Inventors: Philip J. Hartman, Loveland, CO (US); James L. Hartman, Loveland, CO (US)

(73) Assignee: Steam Tech, LLC, Denver, CO (US)

(21) Appl. No.: 16/283,490

(22) Filed: Feb. 22, 2019

(51) Int. Cl. A61M 5/168; A61M 15/00; A61M 5/14

(52) U.S. Cl. CPC A61M 5/16881; A61M 15/08; A61M 5/1409; A61M 15/0086

(57) ABSTRACT

A device, method or kit for delivery of a treatment into a fluid flow for inhalation. More particularly, the invention relates to an infuser having a treatment chamber which receives a treatment or treatment element permeated with a treatment which mixes into a fluid flow to form a treatment vapor which egresses from the infuser.

### I. FIELD OF THE INVENTION

A device, method and kit for delivery of a treatment into fluid flow for inhalation. More particularly, the invention relates to an infuser having a passageway or a treatment chamber which receives a treatment or a treatment element permeated with a treatment which mixes into a fluid flow to form a treatment vapor which egresses from the infuser.

### II. SUMMARY OF THE INVENTION

A broad object of particular embodiments includes an infuser which comprises a housing having a passageway extending between a housing first port and a housing second port including a treatment disposed in the passageway, whereby a fluid passing through the passageway between the housing first port and the housing second port mixes with the treatment to form a treatment vapor which egresses from the second port.

Another broad object of particular embodiments includes an infuser which includes a housing having a first end cap rotatably coupled to a housing first end and a second end cap coupled to a housing second end, wherein the housing has a passageway partitioned to afford a fluid flow passage extending through the housing and a treatment chamber extending through the housing, wherein the first end cap has an internal face which upon rotation of the first end cap fluidically couples the fluid flow passage or the treatment chamber to a fluid source, and wherein a treatment disposed in the treatment chamber mixes with a fluid passing through the treatment chamber to afford a treatment vapor.

Another broad object of particular embodiments of the invention can be to provide a kit to retrofit an inhalation device, wherein the kit comprises an infuser configure to be disposed in a fluid supply line of the inhalation device, wherein the infuser includes one or more of: a housing having a first end cap rotatably coupled to a housing first end and a second end cap coupled to a housing second end, wherein the housing has a passageway partitioned to afford a fluid flow passage extending through the housing and a treatment chamber extending through the housing, wherein the first end cap has an internal face which upon rotation of the first end cap fluidically couples the fluid flow passage or the treatment chamber to a fluid source, and wherein a treatment disposed in the treatment chamber mixes with a fluid passing through the treatment chamber to deliver a treatment vapor to the inhalation device.

Another broad object of particular embodiments of the invention can be method of retrofitting an inhalation device, such as a nasal cannula or a continuous positive airway pressure mask, to include embodiments of an infuser, wherein the method includes dividing a fluid supply conduit to afford a first conduit portion and a second conduit portion and joining the first conduit portion to an first end cap of an infuser and joining the second conduit portion to a second end cap of an infuser.

Another broad object of particular embodiments of the invention can be a method of delivering a treatment vapor to an inhalation device, such as a nasal cannula or a continuous positive airway pressure mask, including one or more of flowing a fluid from a fluid source to through an embodiment of an infuser to the inhalation device, wherein the method can further include disposing a treatment in the treatment chamber of the infuser and flowing the fluid through the treatment chamber of the infuser containing a treatment to deliver a treatment vapor to the inhalation device, and wherein the method can further include treating a disease or disorder condition or alleviating symptoms of a disease or disorder condition with the treatment vapor.

Naturally, further objects of the invention are disclosed throughout other areas of the specification, drawings, photographs, and claims.

### IV. BRIEF DESCRIPTION OF THE DRAWINGS

FIG. 1 is a perspective view of an embodiment of an infuser.

FIG. 2 is an exploded of an embodiment of the infuser shown in FIG. 1.

FIG. 3 is a first end view of an embodiment of the infuser.

FIG. 4 is a second end view of an embodiment of the infuser.

FIG. 5 is a first side view of an embodiment of the infuser.

FIG. 6 is second side view an embodiment of the infuser.

FIG. 7 is a top plan view of an embodiment of the infuser.

FIG. 8 is a bottom plan view of an embodiment of the infuser.

FIG. 9A is a cross section 9-9 of the embodiment of the infuser shown in FIG. 7.

FIG. 9B is a cross section 10-10 of the embodiment of the infuser shown in FIG. 8.

FIG. 10 is a perspective view of an embodiment an infuser.

FIG. 11 is an exploded view of the embodiment of the infuser shown in FIG. 10.

FIG. 12 is an exploded first end view of the infuser.

FIG. 13 is an exploded second end view of the infuser.

FIG. 14 illustrates a method of using an embodiment of the infuser with a nasal cannula.

FIG. 15 is an enlarged portion of FIG. 10 showing an embodiment of the infuser used with a nasal cannula.

FIG. 16 illustrates a method of using an embodiment of the infuser with a continuous positive airway pressure mask.

FIG. 17 is a block flow diagram of methods of making and using embodiments of the inventive infuser.

### V. DETAILED DESCRIPTION OF THE INVENTION

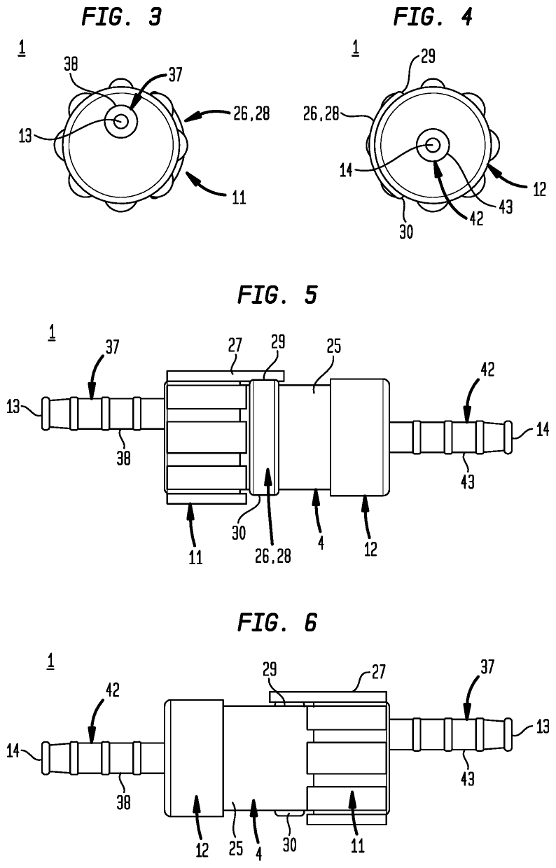

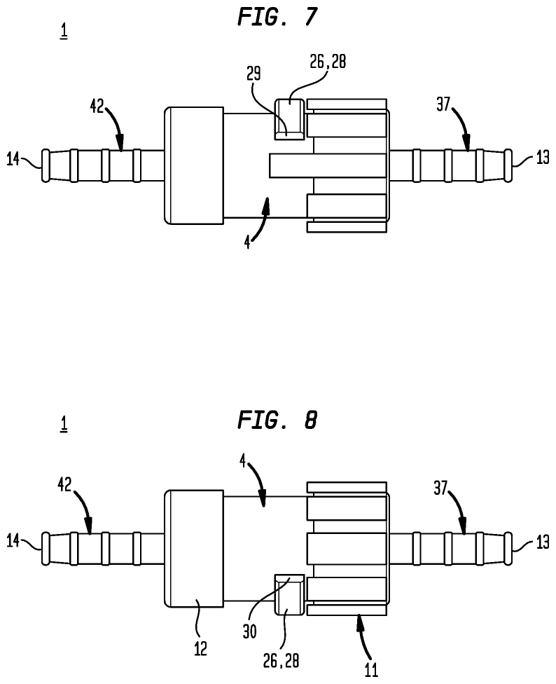

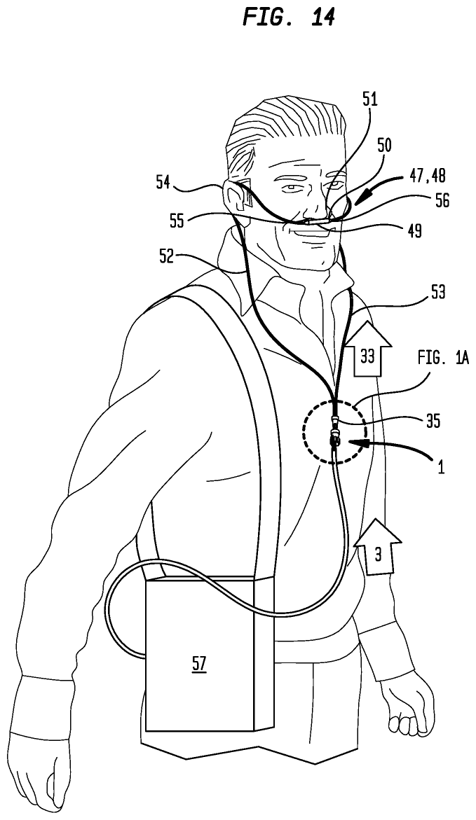

Referring generally to FIGS. 1 through 17, which include embodiments of an infuser (1) and methods of using embodiments of the infuser (1) for infusion of a treatment (2) in a flow of a fluid (3).

Now referring primarily to FIGS. 1 through 8 and 9A and 9B, particular embodiments of the infuser (1) can include a housing (4) having a passageway (5) extending between a housing first end (6) and a housing second end (7). In particular embodiments, a partition (8) can, but need not necessarily, subdivide the passageway (5) into a fluid flow passage (9) extending through the housing (5) and a treatment chamber (10) extending through the housing (4). In particular embodiments, an inlet cap (11) can be rotatingly coupled or removably rotatingly coupled to the housing first end (6) and an outlet cap (12) can be coupled or removably coupled to the housing second end (6). An inlet aperture element (13) can be disposed in the inlet cap (11) and an outlet aperture (14) can be disposed in the outlet cap (12) allowing the fluid (3) to flow through the passageway (5) extending between the housing first end (6) and the housing second end (7).

Again, referring primarily to FIGS. 1 through 8 and 9A and 9B, the outlet cap (12) can include an outlet cap internal face (45) extending to an outlet cap sidewall (45′) including an annular shoulder (45″) which abuts the housing second end (7) to dispose the outlet cap internal face (45) set back from the housing second end (7) to fluidically couple the fluid flow passage (9) and the treatment chamber (10).

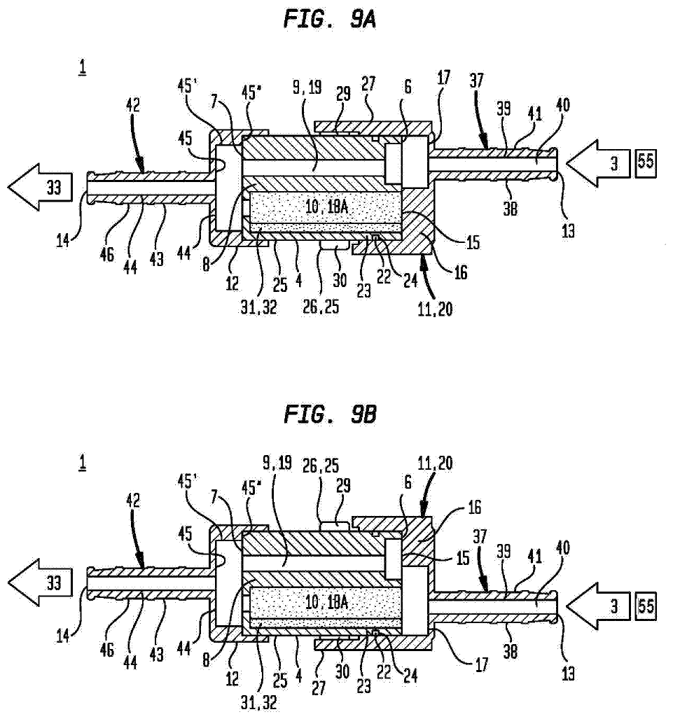

Again, referring primarily to FIGS. 1 through 8 and 9A and 9B, in particular embodiments, the inlet cap (11) can further include an inlet cap internal face (15), including an internal face first portion (16) and an internal face second portion (17). The internal face first portion (16) can abut the housing first end (6) to dispose the treatment chamber (10) at the housing first end (6) in a chamber closed condition (18A) (as shown the example of FIG. 9A) while the internal face second portion (17) sets back from the housing first end (6) to dispose the fluid flow passage (9) in an fluid flow passage open condition (19) (as shown the example of FIG. 9A). In particular embodiments, the housing first end (6) and the internal face first portion (16) can afford generally planar or planar contact surfaces that abuttingly contact to dispose the treatment chamber (10) at the housing first end (6) in a chamber closed condition (18A).

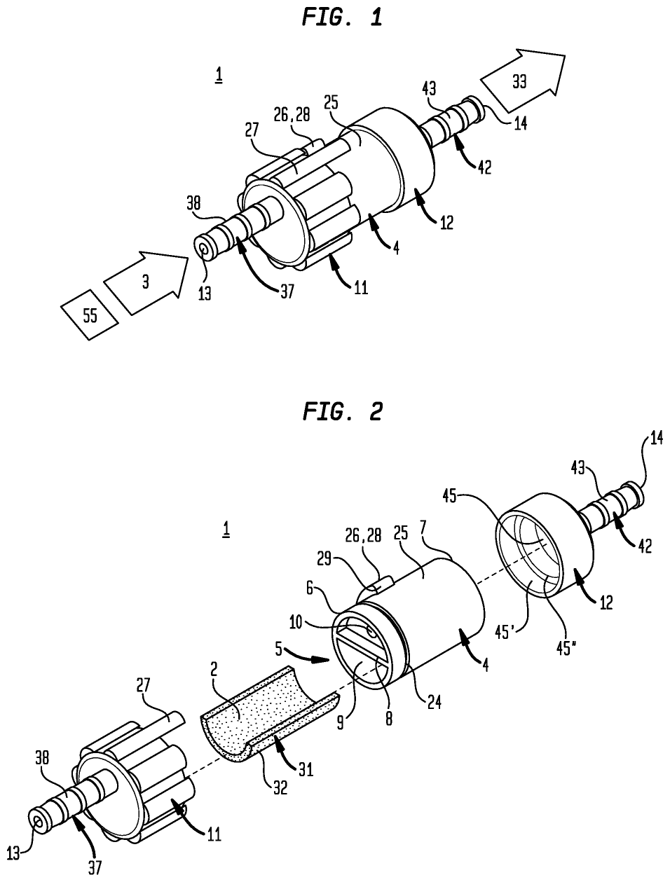

Now, referring primarily to FIGS. 1 and 2 and 5 through 8, in particular embodiments, the inlet cap (11) rotates between an inlet cap first position (20) (as shown in the example of FIG. 9A) and an inlet cap second position (21) (as shown in the example of FIG. 9B). Referring to FIG. 2, in particular embodiments, the inlet cap (11) can include an annular member (22) disposed on an inlet cap internal side wall (23) which can be rotatingly engaged in a corresponding annular groove (24) disposed on a housing external surface (25). Rotating the annular member (22) in the annular groove (24) allows the inlet cap (11) to be rotated between the inlet cap first position (20) and the inlet cap second position (21). In particular embodiments, either one of the annular member (22) or the annular groove (24) need not be circumferentially continuous; rather the annular member (22) can comprise annular segments or detents movable in the annular groove (24) or the annular groove (24) can comprise annular groove segments of sufficient length to allow rotation of the annular segments or detents to rotation of the inlet cap (11) between the first and second positions (20)(21).

Again, referring primarily to FIGS. 1 and 2 and 5 through 8, in particular embodiments, a stop element (26) can, but need not necessarily, be disposed on said housing external surface (25) and a stop engaging element (27) can, but need not necessarily, extend from the inlet cap (11) over the housing external surface (25). The stop engaging element (27) can be responsive to rotation of the inlet cap (11) coupled to the housing first end (6). The stop engaging element (27) engages the stop element (26) to dispose the inlet cap (11) in the first position (20) or the second position (21). In the embodiment shown in FIG. 5, the stop element (26) comprises a raised band (28) circumferentially disposed about the housing (4) having band first and second ends (29)(30) located to respectively engage the stop engaging element (27) to dispose the inlet cap (11) at corresponding first and second positions (20)(21); however, any configuration of raised elements or plurality of raised elements can comprise the stop element (27) to engage the stop engaging element (27) to stop or incrementally stop rotation of the inlet cap (11) between the first and second positions (20)(21).

Now, referring primarily to FIGS. 9A and 9B, the inlet cap (11) can rotate toward the inlet cap first position (20) to abut the internal face first portion (16) with the housing first end (6) to dispose the treatment chamber (10) at the housing first end (6) in a chamber closed condition (18A) while the internal face second portion (17) sets back from the housing first end (6) to dispose the fluid flow passage (9) in an fluid flow passage open condition (19).

Again, referring primarily to FIGS. 9A and 9B, the inlet cap (11) can rotate toward the inlet cap second position (21) in which said internal face first portion (16) abuts said housing first end (6) to dispose said treatment chamber (10) at said housing first end (6) in a chamber open condition (18B) while said internal face second portion (17) sets back from said housing first end (6) to dispose said fluid flow passage (9) in the fluid flow passage open condition (19).

Again, referring primarily to FIGS. 9A and 9B, the inlet cap (11) can be incrementally rotated between the inlet cap first position (20) and the inlet cap second position (21) to correspondingly incrementally dispose the treatment chamber (10) between the chamber closed condition (18A) and the chamber open condition (18B).

Now, referring primarily to FIGS. 2 and 9A, in particular embodiments, the inlet cap (11) can be removed from the housing first end (6) and a treatment element (31) can be disposed in the treatment chamber (10) of the housing (4). In particular embodiments, the treatment element (31) can be impregnated with the treatment (2) and the treatment element (31) impregnated with the treatment (2) can be disposed in the treatment chamber (10) of the housing (4). In particular embodiments, the treatment (2) alone, without the treatment element (31) can be disposed on or in the treatment chamber (10) of the housing (4).

Now, referring primarily to FIG. 2, in particular embodiments, the treatment element (31) can be one or a combination of treatment absorbent material(s) (32). The term “treatment absorbent material (32)” for the purposes of this invention means any material or combination of materials, whether natural or synthetic, capable of retaining or absorbing a treatment (2). The treatment element (32) can be produced by weaving, forming, molding, cutting or other fabrication process to provide a treatment element (32) which can be disposed in the treatment chamber (10) of the housing (6). As illustrative examples, the treatment element (31) can comprise, consist essentially of, or consist of: one or more binders such as gelatin, cellulose, cellulose derivatives, polyvinylpyrrolidone, starch, sucrose or polyethylene glycol; natural or synthetic fibers such as: leather, felt, cotton, wool, cellulose, glass, and plastic, and combinations thereof. As illustrative examples, the treatment element (31) can be variously configured as tubes, sheets, pads, beads, rods, capsules, tablets, granules, particles, or combinations thereof.

Again, referring primarily to FIGS. 2 and 9A, a treatment (2) (shown as stippling on the treatment element (31) in the Figures) can be contained in, disposable on, absorbed by, or retained by a treatment element (31) and disposed in the treatment chamber (10) of the housing (4); however, this is not intended to preclude embodiments in which the treatment (2) without use of a treatment element (31), can be directly disposed in the treatment chamber (10) of the housing (6).

In particular embodiments, a treatment (2) can comprise, consists essentially of, or consist of a volatile substance (33) which evaporates into the fluid (3) contained in or passed through the fluid flow passage (9) extending through said housing (6) or contained in or passed through the treatment element chamber (10) extending through the housing (6). In particular embodiments, the treatment (2) or volatile substance (33) evaporates at temperatures occurring in the range of about −30 degrees F. (about −34 degrees C.) to about 110 degrees F. (about 38 degrees C.); however, this not intended to preclude embodiments in which the treatment (2) can be carried by the fluid (3), as dry particles, droplets or as a vapor or combinations thereof (individually or collectively “a treatment vapor”).

In particular embodiments, the treatment (2) can comprise, consist essentially of, or consist of: a monocyclic hydrocarbon volatile oil, a bicyclic hydrocarbon volatile oil, an ester volatile oil, an ether hydrocarbon volatile oil, an alcohol volatile oil, an aldehyde volatile oil, a ketone volatile oil, an oxide volatile oil, a phenol volatile oil, an essential oil, a terpene, a terpenoid, an alcohol, a vasodilator, a muscle relaxant, an anesthesia gas, a butane gas, a propane gas, a nitrous oxide gas, and a nitrate, and combinations thereof.

Again, referring primarily to FIGS. 9A and 9B, the treatment (2) or the treatment (2) associated with the treatment element (31) can be disposed in the treatment chamber (10) of the housing (6) having the inlet cap (11) rotated toward the inlet cap second position (18B) allowing the fluid (3) to pass through the treatment chamber (10) to mix with the treatment (3) to form a treatment vapor (33) which egresses from said outlet cap (12). For the purposes of this invention the term “fluid” can comprise, consist essentially of, or consist of: a gas and without reduction in the breadth of the foregoing includes as illustrative examples: a mixture of gases, atmospheric gases, air, a purified gas, and oxygen and combinations thereof. For the purposes of this invention the term “treatment vapor” means the treatment (2) mixed with the fluid (3) whether the treatment (2) evaporates into the fluid (3) or the fluid carries the treatment (2) as wet or dry particles, droplets, or vapor, and combinations thereof.

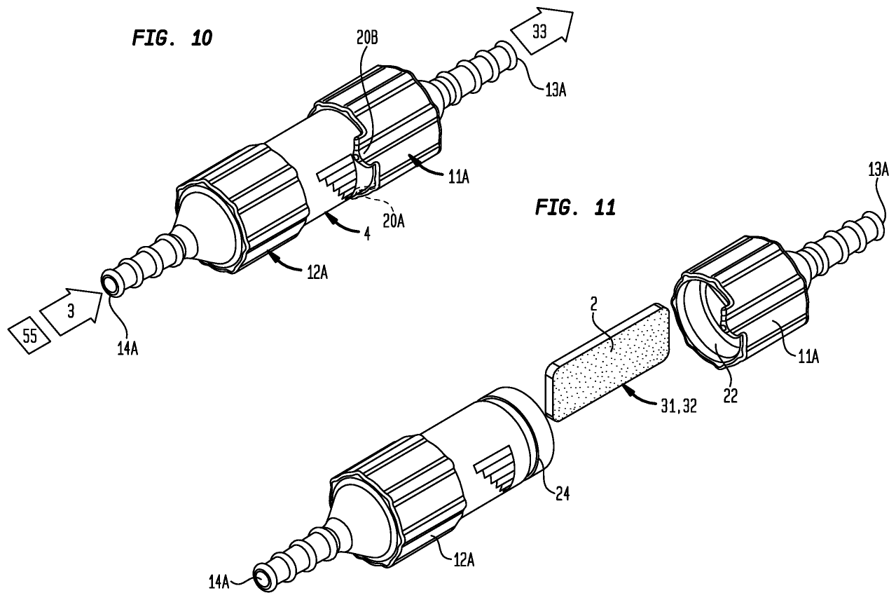

Now referring primarily to FIGS. 10 through 13, in particular embodiments, the infuser (1) can include a housing (4) having a passageway (5) extending between a first port (13A) and a second port (14A). While the Figures show the first and second ports open at opposite ends of the housing (4); this is not intended to preclude embodiments in which the first or second port (13A)(14A) allow ingress or egress of a fluid (3) at the side of housing (4) or both the first and second ports having a location at a housing first end (6) or a housing second end (7).

In the illustrative embodiment of FIGS. 10 through 13, the infuser (1) can include a housing (4) having a passageway (5) extending between a housing first end (6) and a housing second end (7). A partition (8) can subdivide the passageway (5) into a fluid flow passage (9) extending through the housing (5) and a treatment chamber (10) extending through the housing (4). A first end cap (11A) can be rotatingly coupled or removably rotatingly coupled to the housing first end (6) and a second end cap (12A) can be coupled or removably coupled to the housing second end (6). In particular embodiments, the second end cap (12A) can be made one-piece with the housing (4). A first end cap aperture element (13A) can be disposed in the first end cap (11A) and second end cap aperture (14A) can be disposed in the second end cap (12A) allowing the fluid (3) to flow through the passageway (5) and the treatment chamber (10) extending between the housing first end (6) and the housing second end (7).

Again, referring primarily to FIGS. 10 through 13, in particular embodiments, the first end cap (11A) can further include a first end cap internal face (45A), including an internal face first portion (16A) and an internal face second portion (17A). The internal face second portion (17A) can be recessed in relation to the internal face first portion (16A) with the first end cap aperture (13A) communicating between the internal face second portion (17A) and the first end cap end external surface (41A). The internal face first portion (16A) can abut the housing first end (6) and be rotationally disposed to close the fluid flow passage (9) or the treatment chamber (10) or concurrently close the fluid flow passage (9) and the treatment chamber (10). The internal face second portion (17A) can be rotationally disposed to align the recessed internal face second portion (17A) with either the fluid flow passage (9) or the treatment chamber (10) or concurrently both of the fluid flow passage (9) or the treatment chamber (10). Thereby, incremental rotation of the first end cap (11A) between a first position (20A) and a second position (21A) can corresponding incrementally dispose the fluid flow passage (9) between a fluid flow passage open condition (19A) and the fluid flow passage closed condition (19B), and incrementally dispose the treatment chamber (10) between a chamber open condition (18A) and a chamber closed condition (18B), and incremental combinations thereof. The embodiment of the infuser (1) shown in FIGS. 10 through 13 can further include one or more of the components, elements, or features described and shown for the embodiments of the infuser (1) shown in FIGS. 1 through 8 and 9A and 9B and above described.

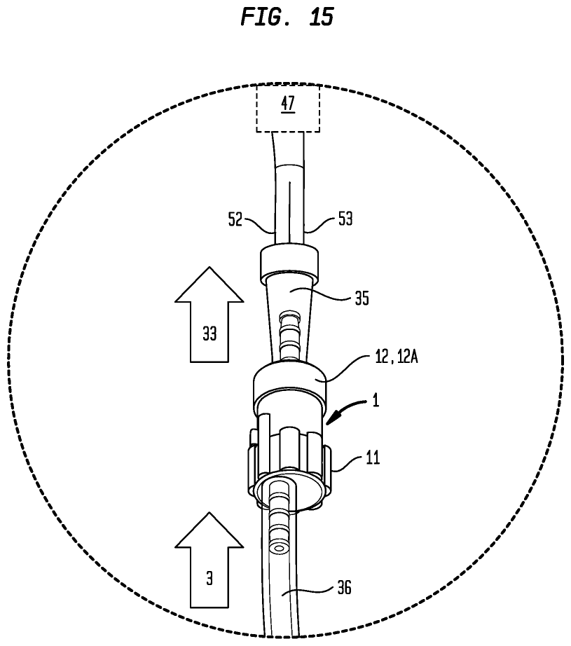

Now, referring primarily to FIG. 15, embodiments of the infuser (1) can, but need not necessarily, be disposed as an integral part of a fluid supply conduit (34). A fluid supply conduit first portion (35) can be coupled to the inlet cap (11) of the infuser (1) and a fluid supply conduit second portion (36) can be coupled to the outlet cap (12) of the infuser (1). In particular embodiments, the inlet cap (11) can include an inlet cap connector (37) for joining the fluid supply conduit first portion (35) to the inlet cap (11). The inlet cap connector (37) can have an inlet cap connector body (38) having any of a wide variety of connector structures to correspondingly join any of wide variety of fluid supply conduit structures; and while the Figures show an inlet cap connector body (38) as having an inlet cap connector body internal surface (39) defining a hollow interior passageway (40) open to an inlet cap internal face (15) and an inlet cap connector body external surface (41) insertable into the fluid supply conduit first portion (35); this is not intended to preclude other embodiments of the inlet cap connector body (38) adapted to or configured to sealably join to the fluid supply conduit first portion (35). As illustrative examples, the inlet cap connector (37) can comprise or consist of: a barbed connector, a barbed stepped connector, a frusto conical connector, a bayonet connector, compression connector, flare connector, funnel connector, luer connector, threaded connector, quick disconnect connector, shielded connector, suction connector, push in connector, or combinations thereof.

Again, referring primarily to FIG. 15, in particular embodiments, the outlet cap (12) can include an outlet cap connector (42) for joining the fluid supply conduit second portion (36) to the outlet cap (12). The outlet cap connector (42) can have an outlet cap connector body (43) having any of a wide variety of connector structures to correspondingly join any of wide variety of fluid supply conduit structures; and while the Figures show an outlet cap connector body (43) having an outlet cap connector body internal surface (44) defining a hollow interior passageway (45) open to an outlet cap internal face (45) and a connector body external surface (46) insertable into the fluid supply conduit second portion (36); this is not intended to preclude other embodiments of the outlet cap connector body (43) adapted to or configured to sealably join to the fluid supply conduit second portion (36). As illustrative examples, the outlet cap connector (42) can comprise or consist of: a barbed connector, a barbed stepped connector, a frusto conical, a bayonet connector, compression connector, flare connector, funnel connector, luer connector, threaded connector, quick disconnect connector, shielded connector, suction connector, push in connector, or combinations thereof. The inlet cap connector (37) and the outlet cap connector (42) can have the same configurations or can have different configurations depending the corresponding configurations of the fluid supply conduit first and second portions (35)(36), which can be the same or different configurations depending on the application.

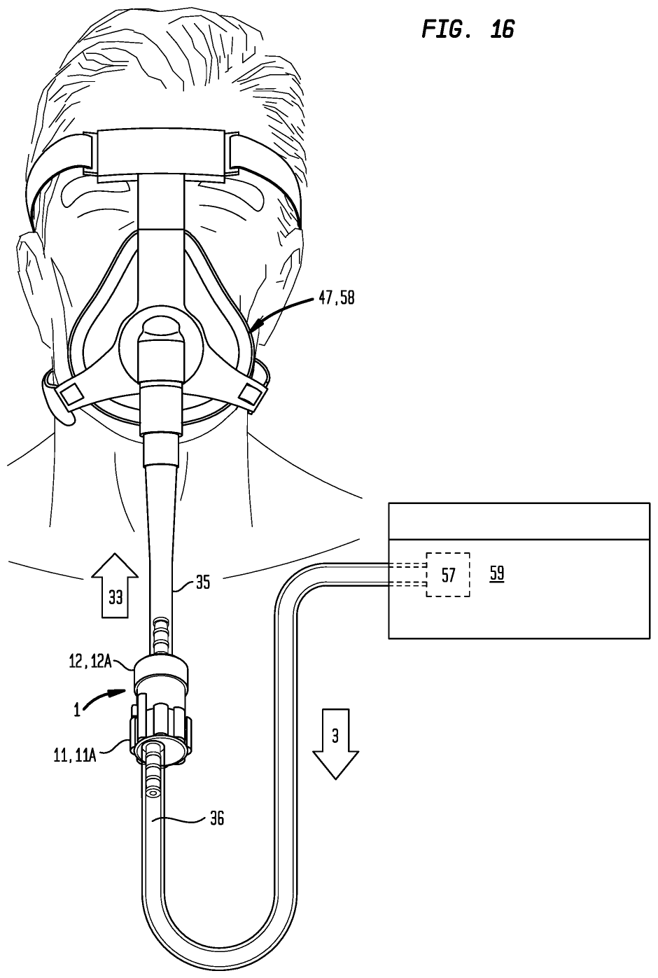

Now, referring primarily to FIGS. 14 and 16, embodiments of the infuser (1) can further include an inhalation device (47). The term “inhalation device” for the purposes of this invention means a device configured to deliver a fluid(s) for inhalation, and without sacrificing the breadth of the forgoing, an inhalation device can deliver a gas or a gas carrying particles, droplets, or vapor for inhalation. The term “inhalation” for the purposes of this invention means drawing a gas or a gas carrying particles, droplets, or vapor into the airways or lungs whether by nasal or oral respiratory route. Illustrative examples of inhalation devices (47) comprise or consist of: nebulizers, compact portable inhalers, metered dose inhalers, nasal cannulas, continuous positive airway pressure devices, anesthesia devices, or combinations thereof.

Now referring primarily to FIG. 14, embodiments of the infuser (1) can further include a nasal cannula (48). Typically, nasal cannulas (48) include a hollow body portion (49) having a pair of spaced-apart elongated tubular portions (50) (nasal interface or nasal prongs) extending from and in fluid communication with the hollow body portion (49). The tubular portions (50) through which a fluid (3) flows can be adapted to fit into the nares (51). A fluid supply conduit first portion (35) can extend from the outlet cap (12) (second end cap (12A) of the infuser (1) and bifurcate into a pair of flexible conduits (52)(53) which can, but need not necessarily, be disposed behind the ears (54) and coupled to opposite ends (55)(56) of the hollow body portion (49) of the nasal cannula (48). A fluid supply conduit second portion (36) can be coupled to the inlet cap (11) (first end cap (11A)) of the infuser (1) and to a fluid source (57) to deliver fluid through the infuser (1) to the nasal cannula (48).

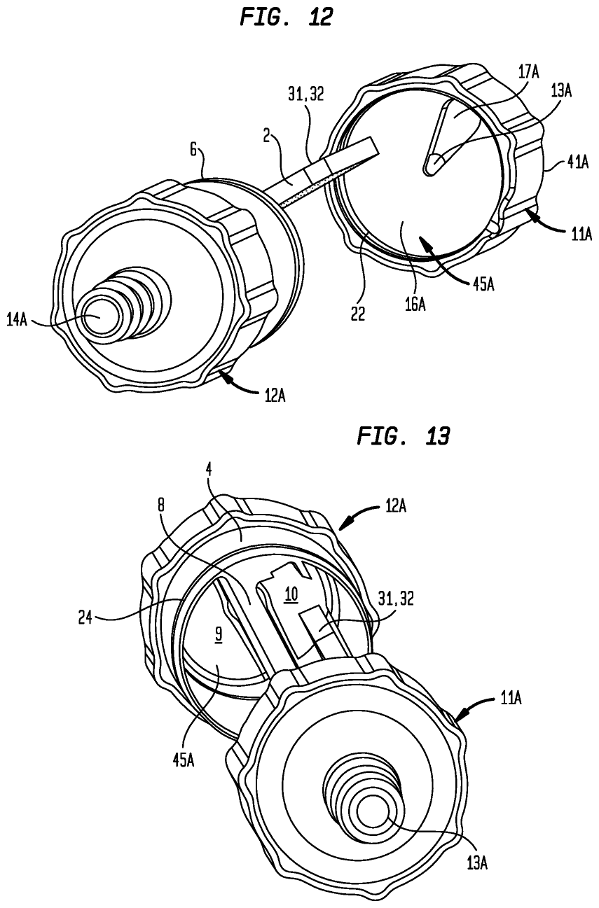

Now referring primarily to FIG. 16, embodiments of the infuser (1) can further comprise a continuous positive airway pressure mask (58) coupled to the outlet cap (12) (or second cap end (12A)) and a continuous positive airway pressure device (59) coupled to the inlet cap (11) (or first end cap (11A)). Continuous positive airway pressure (“CPAP”) comprises a form of positive airway pressure ventilator, which applies mild air pressure on a continuous basis to keep the airways continuously open to stent the lungs' alveoli open and thus recruit more of the lung's surface area for ventilation. Typically, CPAP devices include a mask (58) in the form of a full face mask (as shown in the example FIG. 12), although nasal masks, oral masks, nasal cradle masks, or nasal pillow are also within the scope of the instant invention. A fluid supply line first portion (35) can couple mask (56) to the outlet cap (12) (or second end cap (12A) of the infuser (1). A fluid supply conduit second portion (36) can be coupled to the inlet cap (11) (or first end cap (11A)) of the infuser (1) and to the CPAP device (59) to deliver fluid (3) through the infuser (1) to the mask (58).

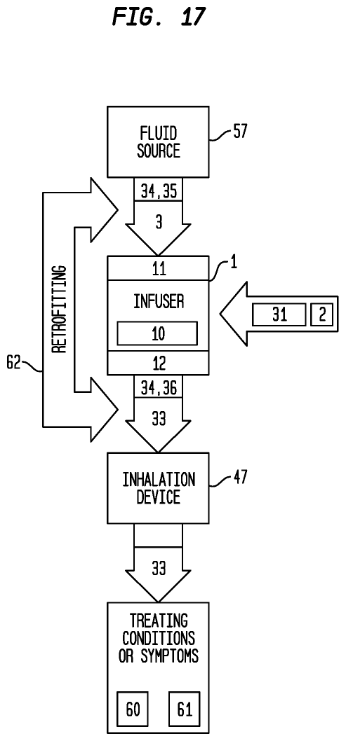

Now referring primarily to FIG. 17, embodiments can include a method including one or more of flowing a fluid (3) from a fluid source (57) to the inhalation device (47). The method can further include disposing a treatment (2) in the treatment chamber (10) of an infuser (1). The method can further include flowing the fluid (3) through the treatment chamber (10) of the infuser (1) containing a treatment (2). The method can further include delivering a treatment vapor (33) to the inhalation device (47) such as a nasal cannula (48) or a continuous positive airway pressure mask (58). The method can further include treating a disease or disorder condition (60) or alleviating symptoms (61) of a disease or disorder condition (60) with the treatment vapor (33).

In particular embodiments, the method can further include retrofitting an inhalation device (47) by disposing embodiments of the infuser (1) as an integral part of a fluid supply conduit (34). The method can further include coupling a fluid supply conduit first portion (35) to the inlet cap (11) (or first end cap 11A) of the infuser (1) and coupling a fluid supply conduit second portion (36) to the outlet cap (12) (or second end cap (12A)) of the infuser (1).

As can be easily understood from the foregoing, the basic concepts of the present invention may be embodied in a variety of ways. The invention involves numerous and varied embodiments of an infuser (1) and methods for making and using such infuser including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “infuser” should be understood to encompass disclosure of the act of “infusing”—whether explicitly discussed or not—and, conversely, were there effectively disclosure of the act of “infusing”, such a disclosure should be understood to encompass disclosure of an “infuser” and even a “means for infusing.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the infusers herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1-35. (canceled)

36. An infuser; comprising
a housing having a passageway extending between a housing first port and a housing second port;
a treatment disposed in said passageway;
a fluid which passes through said passageway between said housing first port and said housing second port, said fluid mixed with said treatment forms a treatment vapor which egresses from said second port.

37. The infuser of claim 36, wherein said fluid comprises a gas.

38. The infuser of claim 37, wherein said fluid is selected from the group consisting of: a mixture of gases, atmospheric gases, a purified gas, and an oxygen gas, and combinations thereof.

39. The infuser of claim 37, wherein said treatment comprises a volatile substance.

40. The infuser of claim 39, wherein said volatile substance evaporates at temperatures occurring in the range of about −30 degrees F. (about −34 degrees C.) to about 110 degrees F. (about 38 degrees C.).

41. The infuser of claim 40, wherein said treatment is selected from the group consisting of: a monocyclic hydrocarbon volatile oil, a bicyclic hydrocarbon volatile oil, an ester volatile oil, ether hydrocarbon volatile oil, an alcohol volatile oil, an aldehyde volatile oil, a ketone volatile oil, an oxide volatile oil, a phenol volatile oil, an essential oil, a terpenoid, an alcohols, a vasodilator, a muscle relaxant, an anesthesia gas, a butane gas, a propane gas, a nitrous oxide gas, and a nitrate, and combinations thereof.

42. The infuser of claim 36, further comprising a treatment element disposable in said passageway, said treatment disposed on said treatment element.

43. The infuser of claim 42, wherein said treatment element comprises a treatment absorbent material.

44. The infuser of claim 43, wherein said treatment absorbent material is select from the group consisting of: leather, felt, cotton fibers, wool fibers, cellulosic fibers, glass fibers, and synthetic fibers, and combinations thereof.

45. The infuser of claim 36, further comprising an end cap coupled to said housing, said first port and second port disposed in said first end cap.

46. The infuser of claim 36, further comprising:
a partition which subdivides said passageway into:
a fluid flow passage extending through said housing between said housing first port and said housing second port; and
a treatment chamber extending through said housing between said housing first port and said housing second port, wherein said treatment disposed in said treatment chamber.

47. The infuser of claim 46, further comprising:
a first end cap coupled to a housing first end, said first port disposed in said first end cap; and
a second end cap coupled to a housing second end, said second port disposed in said second end cap.

48. The infuser of claim 47, wherein said first end cap rotatingly couples to said housing first end, said inlet cap having an inlet cap internal face, including:
an internal face first portion abutting said housing first end; and
an internal face second portion recessed in relation to said internal face first portion;
wherein said first port communicates between said internal face second portion and first end cap external surface.

49. The infuser of claim 48, further comprising a second end cap coupled to said housing second end, said second end cap including:
a second end cap internal face extending to an outlet cap sidewall, said second end cap internal face fluidically coupling said fluid flow passage and said treatment chamber;
wherein said second port communicates between said second cap internal face and a second end cap external surface.

50. The infuser of claim 48, wherein said first end cap rotates toward a first position in which said internal face first portion abuts said housing first end to dispose said treatment chamber at said housing first end in a chamber closed condition while said internal face second portion recessed in relation to said internal face first portion dispose said fluid flow passage in a fluid flow passage open condition.

51. The infuser of claim 50, wherein said first end cap rotates toward a second position in which said internal face first portion abuts said housing first end to dispose said fluid flow passage at said housing first end in a fluid flow passage closed condition while said internal face second portion recessed in relation to internal face first portion disposes said treatment chamber in a chamber open condition.

52. The infuser of claim 51, wherein said inlet cap incrementally rotates between said first position and said second position to correspondingly incrementally dispose said treatment chamber between said chamber closed condition and said chamber open condition.

53. The infuser of claim 51, wherein said inlet cap incrementally rotates between said first position and said second position to correspondingly incrementally dispose said fluid flow passage between said fluid flow passage closed condition and said fluid flow passage open condition.

54. The infuser of claim 50, wherein said treatment element disposable in said a treatment chamber.

55. The infuser of claim 51, wherein said treatment disposed in said treatment chamber having said inlet cap rotated toward said second position allows a fluid to pass through said treatment chamber, said treatment mixed with said fluid to form a treatment vapor which egresses from said outlet cap.

56. The infuser of claim 36, further comprising a nasal cannula coupled to said infuser.

57. The infuser of claim 36, further comprising a continuous positive airway pressure mask coupled to said infuser.
